(19) United States
(12) Patent Application Publication
(10) Pub. No.: US 2012/0022305 A1
(43) Pub. Date: Jan. 26, 2012

(54) HYDROCRACKING PROCESS FOR MAKING RENEWABLE DIESEL FROM OILS AND/OR FATS

(75) Inventors: Jianhua YAO, Bartlesville, OK (US); Dhananjay B. Ghonasgi, Bartlesville, OK (US); Xiaochun Xu, Bartlesville, OK (US); Yun Bao, Bartlesville, OK (US); Edward L. Sughurue, II, Bartlesville, OK (US)

(73) Assignee: CONOCOPHILLIPS COMPANY, Houston, TX (US)

(21) Appl. No.: 13/099,063

(22) Filed: May 2, 2011

Related U.S. Application Data
(63) Continuation-in-part of application No. 11/778,295, filed on Jul. 16, 2007, now Pat. No. 7,955,401.

(51) Int. Cl. C07C 9/02; B01J 19/00; C07C 9/14; C07C 9/22; C07C 1/207; C10G 47/10

(52) U.S. Cl. 585/16; 585/733; 208/112; 422/129

(57) ABSTRACT

Methods for producing C3-C30 hydrocarbons from fatty materials, such as triglyceride compounds, are provided. Hydrocarbon compounds, particularly those boiling in the temperature range of between about 80° F. to about 1000° F., are produced by contacting a fatty material, under hydrocracking conditions, with at least one catalyst comprising a Group VIII metal and a Group VIB metal on a acidic support.

### CROSS-REFERENCE TO RELATED APPLICATIONS

The present invention is a continuation-in-part of U.S. application Ser. No. 11/778,295 filed on Jul. 16, 2007 entitled “HYDROTREATING AND CATALYTIC DEWAXING PROCESS FOR MAKING DIESEL FROM OILS AND/OR FATS”, which is hereby incorporated by reference in its entirety.

### FIELD OF THE DISCLOSURE

The present invention relates generally to methods for converting fatty materials to hydrocarbon-containing product.

### BACKGROUND OF THE DISCLOSURE

As the demand for hydrocarbon fuels increases, the incentives for developing renewable hydrocarbon sources increase as well. Various economic, environmental and political pressures are driving the development of alternative energy sources that are compatible with existing technologies and infrastructure. The development of renewable hydrocarbon fuel sources, such as plant and animal sources has been proposed as a solution to this problem.

“Bio-Diesel” is one such product that may be produced by subjecting a base vegetable oil to a transesterification process using methanol to convert the base oil to desired methyl esters. After processing, the products have very similar combustion properties as compared to petroleum-derived hydrocarbons. However, Bio-Diesel exhibits a number of down sides, especially its poor oxidative stability, propensity to gel in cold climates, and its cost.

Unmodified vegetable oils and fats have also been used as additives in diesel fuel to lower cost and improve the lubricity of the fuel. However, problems such as injector coking and the degradation of combustion chamber conditions have been associated with these unmodified additives. Processes for converting vegetable oil into hydrocarbons have been developed. However, these processes have often involved harsh reaction conditions, or the products from the reaction exhibit undesirable properties (such as high pour and cloud points) which render them unsuitable for use in diesel fuel.

Therefore, a need exists for a process for converting vegetable oils and fats into hydrocarbon compounds in the diesel fuel boiling range which exhibit cold flow properties suitable for use in diesel fuel. Such process should also yield significant quantities of C3-C30 hydrocarbon compounds for improving the cetane rating of diesel fuel.

### BRIEF SUMMARY OF THE DISCLOSURE

This invention relates to a hydrocracking process for converting fatty materials, such as triglycerides, diglycerides, monoglycerides, fatty acids, and combinations thereof to hydrocarbon-containing diesel product compounds exhibiting improved cold flow properties when compared with the diesel products of conventional hydrotreating processes.

Accordingly, in one embodiment, the present invention provides a process comprising the step of contacting a fluid comprising at least one fatty material selected from the group consisting of triglycerides, diglycerides, monoglycerides, free fatty acids and combinations thereof with a hydrocracking catalyst comprising nickel, tungsten and molybdenum on an acidic support. The process occurs under conditions sufficient for converting at least a portion of the at least one fatty material into at least one member selected from the group consisting of C3-C30 hydrocarbons and combinations thereof.

### DETAILED DESCRIPTION OF THE DISCLOSURE

In one embodiment of the present invention, an improved hydrotreating process is provided for converting fatty materials, especially those selected from the group consisting of triglycerides, diglycerides, monoglycerides, free fatty acids, and combinations thereof into C3-C30 hydrocarbons, especially diesel boiling range hydrocarbons.

The process results in a reaction product having enhanced cold flow properties when compared to products produced according to more conventional hydrotreating process.

The present inventive hydrocracking process comprises hydrocracking an organic feed including at least one fatty material using a catalyst comprising a Group VIII metal and a Group VIB metal (of the CAS periodic table) such as nickel, tungsten and molybdenum on an acidic support selected from the group consisting of zeolites, molecular sieves, and any combination thereof. In certain embodiments, the zeolite that forms a part of the hydrocracking catalyst is beta zeolite, Y zeolite or the combination. Hydrocracking catalyst is commercially available and may be obtained from Criterion, Holder Topsoe, Axens, UOP or etc.

The conventional hydrotreating process uses a hydrotreating catalyst which may be any catalyst known in the art to be suitable for hydrotreating operations, especially those catalysts which comprise a Group VIII metal and a Group VIB metal (of the CAS periodic table) on a porous support such as a hydrotreating catalyst comprises cobalt and molybdenum or nickel and molybdenum on an alumina support. The conventional hydrotreating catalyst is commercially available and may be obtained from Haldor Topsoe, Albemarle, Criterion or etc.

A reaction feed comprising at least one fatty material is supplied to the hydrocracking process. As used herein, the term “fatty material” refers to a product that comprises, consists of, or consists essentially of a fatty acid or residue thereof. In certain embodiments, the fatty material is selected from the group consisting of triglycerides, diglycerides, monoglycerides, and free fatty acids. The term “triglyceride” generally refers to a naturally occurring ester of a fatty acid and/or glycerol having the general formula CH₂(OCOR₁)—CH(OCOR₂)CH₂(OCOR₃), where R₁, R₂, and R₃ are the same or different and may vary in chain length. Di- and monoglycerides comprise one or two fewer ester moieties, respectively. In certain embodiments, the fatty material, especially the triglyceride compound, is selected from the group consisting of vegetable oil, yellow grease (such as used restaurant oil or those derived from used cooking oils), animal fats, and mixtures thereof. Exemplary vegetable oils include, but are not limited to soybean oil, corn oil, peanut oil, sunflower seed oil, coconut oil, babassu oil, grape seed oil, poppy seed oil, almond oil, hazelnut oil, walnut oil, olive oil, avocado oil, sesame oil, tall oil, cottonseed oil, palm oil, rice bran oil, canola oil, cocoa butter, shea butter, butyrospermum, wheat germ oil, illipse butter, meadowfoam, seed oil, rapeseed oil, borange seed oil, linseed oil, caster oil, vernoia oil, tung oil, jojoba oil, ongokea oil. Exemplary animal fats include tallow animal fat, beef fat, chicken fat, pork fats, poultry grease, and milk fat.

The reaction feed may also include at least one hydrocarbon compound having a boiling point of between about 80° F. to about 1000° F. Exemplary hydrocarbon compounds include middle distillate fuels. Middle distillate fuels generally contain hydrocarbons that boil in the middle distillate boiling range of between about 300° F. to about 750° F. Typical middle distillate fuels include those selected from the group consisting of gasoline, naphtha, jet fuel, kerosene, diesel fuel, light cycle oil (LCO), vacuum gas oil, atmospheric gas oil, atmospheric tower bottoms, and combinations thereof. In one embodiment, the middle distillate fuel presents an API gravity (ASTM D287) of between about 20 to about 50. In addition, the middle distillate fuels present a minimum flash point (ASTM D93) of greater than about 80° F., and in other embodiments, greater than about 90° F.

Hydrocarbon compounds present in the reaction feed may also contain a quantity of aromatics, olefins, and sulfur, as well as paraffins and naphthenes. The amount of aromatics in the hydrocarbon generally may be in an amount of between about 0 to about 100 weight % based on the total weight of the hydrocarbons. In one embodiment, aromatics are present in an amount of between about 20 to about 80 weight %. The amount of olefins in the hydrocarbon generally may be in an amount of less than about 10 weight % based on the total weight of the hydrocarbon. In one embodiment, the olefins are present in an amount of less than about 5 weight %, and in still another embodiment, olefins are present in an amount of less than about 2 weight %.

The amount of sulfur in the hydrocarbon can generally be greater than about 50 parts per million by weight (ppmw). In one embodiment, sulfur is present in an amount of between about 100 ppmw to about 50,000 ppmw, and in another embodiment, sulfur is present in an amount of between about 150 to about 4,000 ppmw. As used herein, the term “sulfur” denotes elemental sulfur, and also any sulfur compounds normally present in a hydrocarbon stream, such as diesel fuel. The catalysts used with the present invention may also serve to remove sulfur compounds present in the hydrocarbon portion of the reaction feed. Exemplary sulfur compounds which may be removed include, but are not limited to, hydrogen sulfide, carbonyl sulfide (COS), carbon disulfide, mercaptans (RSH), organic sulfides (R-S-R), organic disulfides R-S-S-R), thiophene, substituted thiophenes, organic trisulfides, organic tetrasulfides, benzothiophene, alkyl thiophenes, dibenzothiophene, alkyl benzothiophenes, alkyl dibenzothiophenes, and mixtures thereof, as well as heavier molecular weights of the same, wherein each R can be an alkyl, cycloalkyl, or aryl group containing about 1 to about 10 carbon atoms.

The reaction feed generally comprises between about 0.1 to about 99.9 weight % fatty materials, based on the total weight of the feed. In other embodiments, the feed comprises between about 2 to about 80 weight % fatty material, and in still other embodiments, the feed comprises between about 5 to about 30 weight % fatty materials. If present, the hydrocarbon compound generally comprises between about 0.1 to about 99.9 weight % of the feed. In other embodiments, the feed comprises between about 10 to about 98 weight % of the hydrocarbon compound, and in still other embodiments, the feed comprises between about 50 to about 95 weight % of the hydrocarbon compound. In certain embodiments, the weight ratio of fatty material to hydrocarbon in the reaction feed is between about 1:1000 to about 1000:1. In other embodiments, the weight ratio of fatty material to hydrocarbon in the reaction feed is between about 1:50 to about 50:1. In still other embodiments, the weight ratio of fatty material to hydrocarbon in the reaction feed is between about 1:25 to about 25:1.

The reaction feed is contacted with the hydrocracking catalyst within a reaction zone under conditions sufficient for converting at least a portion of the at least one fatty material present in the feed into a member selected from the group consisting of C3-C30 hydrocarbons and combinations thereof. The reaction zone may comprise any suitable reactor that enables intimate contact of the reactants and control of the operating conditions. Exemplary reactors include fixed bed reactors and fluidized bed reactors. As used herein, the term “fluidized bed reactor” denotes a reactor wherein a fluid feed can be contacted with solid particles in a manner such that the solid particles are at least partly suspended within the reaction zone by the flow of the fluid feed through the reaction zone and the solid particles are substantially free to move about within the reaction zone as driven by the flow of the fluid feed through the reaction zone.

Generally, the reaction zone is maintained at a temperature of between about 500° F. to about 800° F., and in certain embodiments, at a temperature of between about 700° F. to about 800° F. The pressure in the reaction zone is generally between about 100 psig to about 2500 psig. In one embodiment employing a fixed bed reactor, the pressure is maintained between about 100 psig to about 2500 psig. In one embodiment employing a fluidized bed reactor, the pressure is maintained between about 400 psig to about 2500 psig.

As used herein, the term “liquid hourly space velocity” or “LHSV” refers to the numerical ratio of the rate at which the reactants are charged to the reaction zone in barrels per hour at standard conditions of temperature and pressure (STP) divided by the barrels of catalyst contained in the reaction zone to which the reactants are charged. In certain embodiments, the LHSV is between about 0.2 hr⁻¹ to about 5 hr⁻¹. In other embodiments, the LHSV is between about 0.5 hr⁻⁻¹ to about 5 hr⁻¹. In still other embodiments, the LHSV is between about 1.0 hr⁻¹ to about 3.0 hr⁻¹.

The reaction product generally comprises gas and liquid fractions containing hydrocarbon products, which include, but are not limited to, diesel boiling range hydrocarbons. The reaction product generally comprises long chain carbon compounds having 3-30 or more carbon atoms per molecule, especially those selected from the group consisting of C3-C30 hydrocarbons and combinations thereof. In certain embodiments, the reaction product comprises between about 0.1 to about 99.9 weight % of C3-C30 hydrocarbons. In other embodiments, the reaction product comprises between about 10 to about 98 weight % of C3-C30 hydrocarbons. In addition, the reaction product can further comprise by-products of carbon monoxide, carbon dioxide, and propane.

Generally, the hydrocarbon products of the hydrocracking process have a sulfur content that is substantially less than the sulfur content present in the reaction feed. The sulfur content of the product is at least 25% less than the sulfur content present in the reaction feed. In another embodiment, the sulfur content of the product is at least 50% less than the sulfur content present in the reaction feed. In still another embodiment, the sulfur content of the product is at least 75% less than the sulfur content present in the reaction feed.

The cetane number of the diesel range hydrocarbon product is determined using ASTM test method D 613.65. For example, the cetane number for a light cycle oil (LCO) feed stock is typically less than 28, and may in some instances be less than 26 or less than 24. Generally, the cetane number of the hydrocarbon product produced in accordance with the present invention will have a cetane number greater than that of the original feedstock.

The pour point of the diesel range hydrocarbon product is determined using ASTM test method D 97. Generally, the pour point is the lowest temperature at which a petroleum product will begin to flow. The improvement of pour point by using hydrocracking catalyst compared with hydrotreating catalyst various depended on the feed composition and operating conditions. In one embodiment where the feed contains 20% oil/fat and 80% light vacuum gas, the improvement of pour point will be at least 5° F.

The cloud point of the hydrocarbon product is determined using ASTM test method D 2500. Generally, the cloud point is the temperature at which dissolved solids, such as wax crystals, begin to form in a petroleum product as it is cooled. In one embodiment, the hydrocarbon product of the hydrocracking process present cloud points of less than about 20° F. In one embodiment, the hydrocarbon product presents a cloud point of between about −20° F. to about 10° F. In another embodiment, the hydrocarbon product presents a cloud point of between about −15° F. to about 5° F. In still another embodiment, the hydrocarbon product presents a cloud point of between about −12° F. to about 0° F. In yet other embodiments, the hydrocarbon product presents slightly higher cloud points of between about 10° F. to about 20° F. In certain embodiments, the cloud point of the hydrocarbon product of the current hydrocarcking process is at least 20% lower than which of the conventional hydrotreating process under the same temperature, pressure and space velocity condition. In yet another embodiment, the cloud point of the hydrocarbon product of the current hydrocracking process is at least 30% lower than which of the conventional hydrotreating process under the same temperature, pressure and space velocity condition.

The liquid yield (v/v) of the hydrocracking process is generally between about 0.60 to about 1.0. In one embodiment, the liquid yield is between about 0.75 to about 0.99, and in another embodiment, the liquid yield is between about 0.8 to about 0.98. In yet another embodiment, the liquid yield is between about 0.85 to about 0.95.

The liquid yield of the hydrocracking process may be improved without significantly impairing the cold flow properties of the reaction product using a modified zeolite catalyst, particularly a modified Y zeolite or beta zeolite. The process of forming the modified catalyst begins with one or more treatment steps on the zeolite support for the catalyst. The zeolite support, prior to depositing of the cobalt and molybdenum thereon, undergoes an acid leaching step and/or a steam treatment step. In the acid leaching step, the zeolite, which comprises at least some alumina as a binder, is contacted with an acid for a sufficient period of time to remove (or leach out) at least a portion of the alumina present therein. In one embodiment, the acid used is hydrochloric acid, however any strong acid capable of leaching out the alumina may be used. In one embodiment, the zeolite support is contacted with the acid at a temperature of between about 70° C. to about 100° C. for between about 1 to about 3 hours. Thus, the acid leaching step removes significant amounts of alumina from the zeolite so that the end catalyst comprises very little, or substantially no alumina. The catalyst formed using the modified zeolite support comprises less than about 10 weight % alumina. In one embodiment, the modified zeolite catalyst comprises less than about 5 weight % alumina; and in yet another embodiment, the modified zeolite catalyst comprises less than about 1 weight % alumina.

After the leaching process, the acid is removed from the support and washed, such as with distilled water, to remove any acid residues. The zeolite support may then undergo a steam treatment process during which it is contacted with steam at a temperature of between about 400° C. to about 750° C. In one embodiment, the support is contacted with steam at a temperature of between about 500° C. to about 700° C. The steam treatment step may last for at least about 2 hours, and in one embodiment, for between about 2 to about 6 hours. The cobalt and molybdenum is then loaded onto the zeolite support in much the same way as would occur during the preparation of the above-described Co/Mo on zeolite catalyst. The zeolite support is contacted with cobalt and molybdenum compounds in solution. In one embodiment, the solutions comprise ammonium molybdate and cobalt nitrate. The catalyst is then dried and calcined. In one embodiment, calcining occurs at a temperature of between about 400° C. to about 600° C., and in another embodiment at a temperature of between about 425° C. to about 500° C. The calcining may last for up to about 8 hours, however, in one embodiment, the calcining step lasts for between about 3-7 hours.

The hydrocracking conditions using the modified zeolite catalyst are the same as those described above. The product of the hydrocracking process is also very similar to that described above. The cold flow properties of the hydrocarbon product may be slightly less favorable, but still within acceptable ranges and a vast improvement over conventional hydrotreating technologies. Further, a much greater liquid yield can be achieved.

### Example 1

In this example, one conventional hydrotreating catalyst and one hydrocracking catalyst were prepared and tested for comparison study. The cold flow properties of the resulting diesel fuel blends were determined.

The catalyst 1 was a commercially available hydrotreating catalyst under the designation TK-574 from Haldor Topsoe, Inc., Houston, Tex. The catalyst 2 was also a commercially available hydrocracking catalyst.

A mixture comprising 20 wt % Yellow Grease and 80 wt. % Light Volume Gas Oil was contacted with catalysts 1-2 under the same conditions (750° F., at 1300 psig, at a liquid hourly space velocity of 2 hr⁻¹). The cold flow properties of the resulting products were determined and are shown in Table 1.

TABLE 1

| | Catalyst | |
|---|---|---|
| | Hydrotreating | Hydrocracking |
| | Catalyst | Catalyst |
| Temp. F. | 750 | |
| Pressure, F | 1300 | |
| Space Velocity, hr⁻¹ | 2 | |
| Feed Composition | 20% Yellow Grease + 80% Borger | |
| | Refinery Light Vacuum gas Oil | |
| Diesel Yield in the product, wt % | 43 | 53 |
| Diesel product properties | | |
| Sulfur, ppm | 200 | 190 |
| Cetane No. | 59.9 | 57.2 |
| Cloud Point, F | 44 | 28 |
| Pour Point, F | 42 | 27 |
| Density, g/ml | 0.819 | 0.805 |

As illustrated in Table 1, by contacting the reactor feed with hydrocracking catalysts, the cold flow properties of the diesel product from the reaction can be improved and the sulfur content of the diesel product decreased when compared to the diesel products of a conventional hydrotreating process employing the hydrotreating catalyst. The product formed from catalyst 2 (hydrocracking catalyst) exhibited much higher diesel yield and much better cold flow properties of the diesel product than which were formed from catalyst 1 (conventional hydrotreating catalyst).

The embodiments of the invention described above are to be used as illustration only, and should not be used in a limiting sense to interpret the scope of the present invention. Obvious modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

What is claimed is:

1. A process comprising:
a) providing a fluid feed stream comprising at least one fatty material selected from the group consisting of triglycerides, diglycerides, monoglycerides, and free fatty acids;
b) providing a hydrocracking catalyst comprising a Group VIII metal and a Group VIB metal on an acidic support; and
c) contacting said fluid feed stream with said catalyst under conditions sufficient for converting at least a portion of said at least one fatty material into at least one member selected from the group consisting of C3-C30 hydrocarbons and combinations thereof.

2. A renewable fuel range hydrocarbon produced by:
a) providing a fluid comprising at least one fatty material selected from the group consisting of triglycerides, diglycerides, monoglycerides, and free fatty acids;
b) providing a hydrocracking catalyst comprising a Group VIII metal and a Group VIB metal on an acidic support; and
c) contacting said fluid with said catalyst under conditions sufficient for converting at least a portion of said at least one fatty material into at least one member selected from the group consisting of C3-C30 hydrocarbons and combinations thereof.

3. A system comprising:
a) a fluid feed stream comprising at least one fatty material selected from the group consisting of triglycerides, diglycerides, monoglycerides, and free fatty acids;
b) a hydrocracking catalyst comprising a Group VIII metal and a Group VIB metal on an acidic support; and
c) a reactor for reacting said fluid feed stream with said catalyst conditions sufficient for converting at least a portion of said at least one fatty material into at least one member selected from the group consisting of C3-C30 hydrocarbons and combinations thereof

4. The process according to claim 1,
wherein said acidic support is selected from the group consisting of zeolites, molecular sieves, alumina-silica, and combinations thereof and
wherein said hydrocracking catalyst comprises nickel, tungsten and molybdenum.

5. The process according to claim 4, wherein said hydrocracking catalyst comprises nickel, tungsten and molybdenum on a zeolite support.

6. The process according to claim 4, wherein said zeolite support comprises beta zeolite or Y zeolite.

7. The process according to claim 1, wherein said fluid, prior to being contacted with said catalyst, comprises at least one feed hydrocarbon having a boiling point of between about 80° F. to about 1000° F.

8. The process according to claim 7, wherein said at least one feed hydrocarbon is selected from the group consisting of gasoline, naphtha, jet fuel, kerosene, diesel fuel, light cycle oil (LCO), vacuum gas oil, atmospheric gas oil, atmospheric tower bottoms, and combinations thereof.

9. The process according to claim 7, wherein said at least one feed hydrocarbon is present in said fluid at a level of between about 0.1 to about 99.9 weight %.

10. The process according to claim 7, wherein the weight ratio of said at least one fatty material to said at least one feed hydrocarbon is between about 1:1000 to about 1000:1.

11. The process according to claim 1, wherein said fluid comprises between about 0.1 to about 99.9 weight % of said at least one fatty material.

12. The process according to claim 1, wherein said fluid, after being contacted with said catalyst, presents a cloud point of below about −20° F.

13. The process according to claim 1, wherein said fluid, after being contacted with said catalyst, presents a pour point of between −20° F. and 10° F.

14. The process according to claim 1, wherein said contacting step is performed at a pressure of between about 100 psig to 2500 psig and at a temperature of between about 500° F. to about 800° F.

15. The process according to claim 1, wherein said fluid, after said contacting step, comprises between about 0.1 to about 99.9 weight % of C3-C30 hydrocarbons.

16. The process according to claim 1, wherein said fatty material is selected from the group consisting of vegetable oil, yellow grease, animal fats and mixtures thereof
